(19) United States
(12) Patent Application Publication
(10) Pub. No.: US 2017/0266431 A1
(43) Pub. Date: Sep. 21, 2017

(54) ELECTROTHERAPEUTIC GARMENT

(71) Applicant: WEARTECH THERAPEUTICS, INC., Gainesville, GA (US)

(72) Inventor: James P. JOHNSON, Gainesville, GA (US)

(73) Assignee: WEARTECH THERAPEUTICS, INC., Gainesville, GA (US)

(21) Appl. No.: 15/464,554

(22) Filed: Mar. 21, 2017

Related U.S. Application Data
(60) Provisional application No. 62/311,056, filed on Mar. 21, 2016.
(60) Provisional application No. 62/334,676, filed on May 11, 2016.

(51) Int. Cl. A61N 1/04; A61N 1/36

(52) U.S. Cl. CPC A61N 1/0484; A61N 1/36014; A61N 1/0492; A61N 1/048; A61N 1/0452; A61N 1/36003

(57) ABSTRACT

An electrotherapeutic garment, for example in the form of pants, having at least one electrode portion and an electrically conductive tracer ribbon embedded in the garment for connecting the electrode portion(s) to an electro-stimulation device.

### CROSS-REFERENCE TO RELATED APPLICATION

This application claims the benefit of U.S. Provisional Patent Application Ser. No. 62/311,056 filed Mar. 21, 2016 and U.S. Provisional Patent Application Ser. No. 62/334,676 filed May 11, 2016, the entireties of which are hereby incorporated herein by reference for all purposes.

### TECHNICAL FIELD

The present invention relates generally to the field of wearable garments, and more particularly to an electrotherapeutic garment.

### BACKGROUND

Providing continuous, variable DC electrotherapeutic energy to the body part of a patient can increase microcirculation to the designated body part resulting in better pain management, a reduction of disuse atrophy (muscle wasting), and relief of muscle spasticity, edema (swelling), and inflammation which all contributes to improve rapid soft tissue recovery and enhance endurance. This therapy can be particularly effective when the patient is sleeping. While sleeping, the human body's biological healing cycle is more regenerative and pronounced when 70% of growth hormones are produced along with Venous Endothelia Growth Factor. Further, pain is often more pronounced at night and pain relief can allow the patient to sleep better. Lastly, delivering therapy during sleep allows the patient to receive a longer course of treatment. Therefore, there exists a need for a garment that delivers electrotherapy that is comfortable to wear while sleeping.

U.S. patent application Ser. No. 10/862,120 of James P. Johnson, filed Jun. 4, 2004 (U.S. Patent Pub. No. 2004/0254624 A1) is hereby incorporated herein by reference.

### SUMMARY

In one aspect, example embodiments of the present invention relate to wearable garments such as for example pants, leggings, tights, trousers, shorts, shirts, athletic wear, and knee, elbow and ankle sleeves, and the like, for providing electrotherapeutic energy to one or more body parts of a human or animal wearer of the garment.

In another aspect, the invention relates to an electrotherapeutic garment for delivering electrotherapy to the body part of a human or animal patient including at least one portion of stimulating material, a garment material configured to hold the stimulating material in contact with the body part of the patient, at least one stimulating device configured to produce electrotherapeutic energy, and at least one conductive tracer configured to transfer the electrotherapeutic energy produced by the at least one stimulating device to the at least one portion of electrotherapeutic material.

In still another aspect, the invention relates to an electrotherapeutic garment for delivering electrotherapy to the body part of a human or animal patient including at least one portion of stimulating material formed from silver conductive material, a garment material and at least one stimulating device configured to produce electrotherapeutic energy.

In still another aspect, the invention relates to a method of treating a body portion of a human or animal subject including having the subject wear an electrotherapeutic garment over the body portion to be treated and delivering therapeutic energy to the body portion through the electrotherapeutic garment. In further examples, the electrotherapeutic garment is used while the subject is sleeping, for example in the form of pajamas or other sleep attire configured for comfort while lying in a horizontal, reclined or recumbent position.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1A shows a therapeutic tights garment according to an example embodiment of the invention.

FIG. 1B shows the therapeutic tights garment of FIG. 1 without attached stimulator devices.

FIG. 2 shows the inside of the therapeutic tights garment of FIG. 1.

FIG. 3 shows the inside of the therapeutic tights garment of FIG. 1.

FIG. 4 shows a therapeutic tights garment according to another example embodiment of the invention.

FIG. 5 shows a therapeutic tights garment according to another example embodiment of the invention.

FIG. 6 shows a therapeutic shirt garment according to an example embodiment of the invention.

FIG. 7 shows the inside of the therapeutic shirt garment of FIG. 7.

FIG. 8 shows a therapeutic knee sleeve garment according to an example embodiment of the invention.

FIG. 9 shows the inside of the therapeutic knee sleeve garment of FIG. 9.

FIG. 10 shows a therapeutic ankle sleeve garment according to an example embodiment of the invention.

FIG. 11 shows the inside of the therapeutic ankle sleeve garment of FIG. 11.

FIG. 12 shows a therapeutic elbow sleeve garment according to an example embodiment of the invention.

FIG. 13 shows the inside of the therapeutic elbow sleeve garment of FIG. 13.

FIG. 14 shows a therapeutic forearm sleeve garment according to an example embodiment of the invention.

FIG. 15 shows the inside of the therapeutic forearm sleeve garment of FIG. 15.

FIG. 16 shows a therapeutic leg sleeve garment according to an example embodiment of the invention.

FIG. 17 shows the inside of the therapeutic leg sleeve garment of FIG. 17.

FIG. 18 shows a therapeutic leg sleeve garment for use on an animal patient according to an example embodiment of the invention.

FIG. 19 shows a therapeutic face mask garment according to an example embodiment of the invention.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

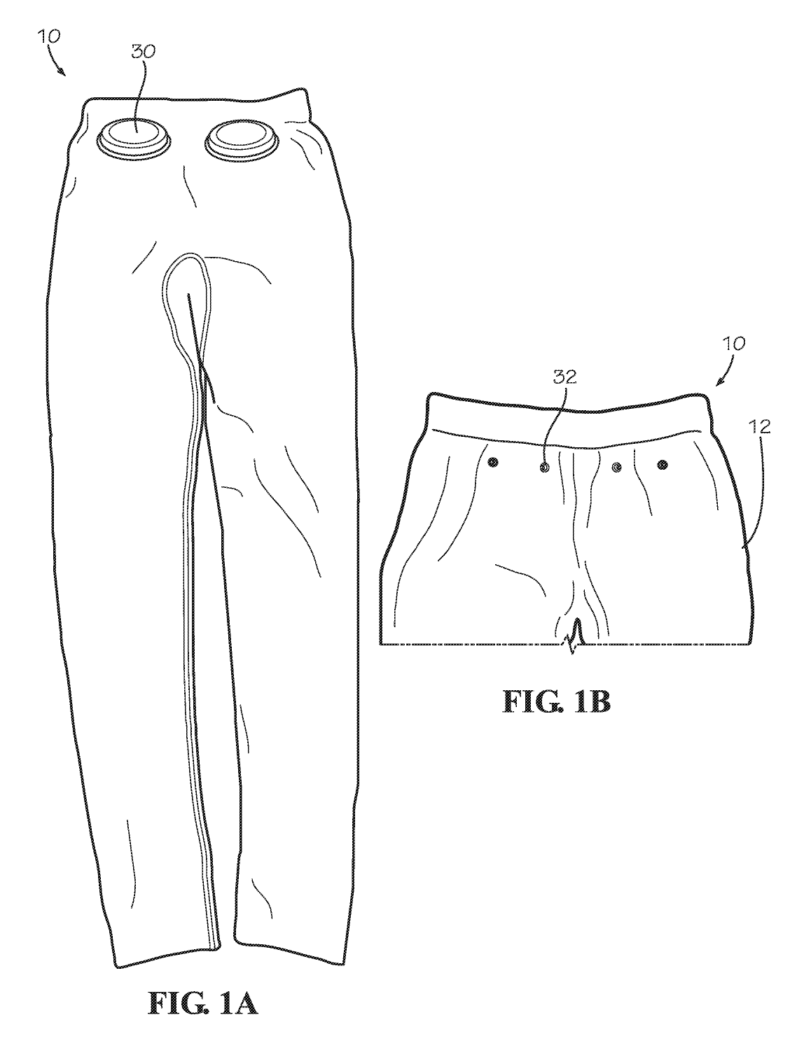

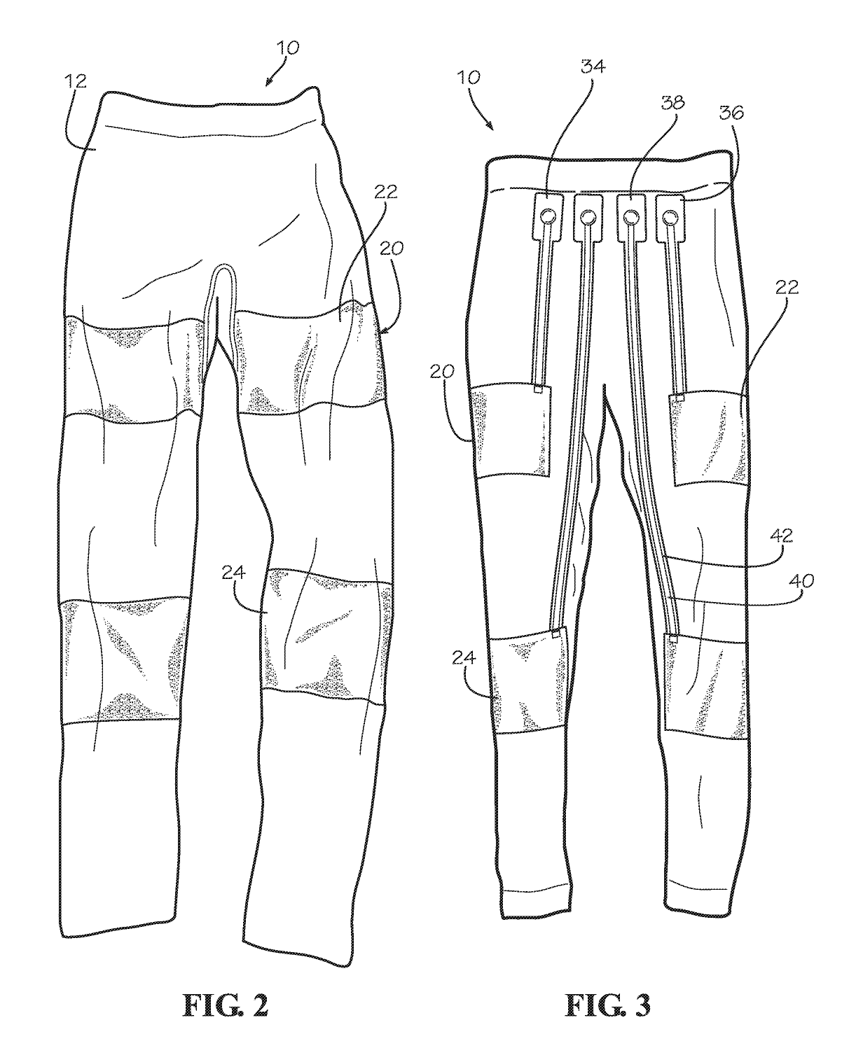

With reference now to the drawing figures, wherein like reference numbers represent corresponding parts throughout the several views, FIGS. 1-20 show therapeutic garments according to example embodiments of the invention. The therapeutic garments are configured to be worn on a portion of the body of a human or animal patient. The garments are configured to provide continuous, variable DC electrotherapeutic energy to an exterior surface of the body of the patient. The garments generally include a stimulator device for generating DC energy and a stimulator material that conducts the energy against a body part of the patient.

In example embodiments, the garments can be constructed of spandex, lycra, cotton, nylon, polyester, polypropylene, tencel, wool, or any other fabric or combination of fabrics suitable to form a generally tight skin contact with the treatment area of the patient. In example embodiments, the garment is made of a woven material. The garment material can also be flexible and washable. The garment can be configured to provide electrotherapy to an exterior surface of the body of the patient including the legs, hands, feet, elbows, knees or face. The garments can include leg wear, shirts, stockings, gloves, masks and various panels and wraps.

The stimulator device is configured to deliver electrical stimulation from an energy source such as a battery. In example embodiments, the stimulator device includes a housing configured to hold batteries or other energy source. The housing generally includes terminals or contacts to create an electrically conductive connection. The housing can also include a mechanism for user input such as an on/off switch or button and a voltage adjustment switch or button. In example embodiments, the stimulator device is configured for high voltage, low amperage pulsed galvanic stimulation with an intensity amplitude ranging from about 0 to about 200 volts. The stimulator device can be configured for a pulse rate of about 1 to about 120 pulses per second. The voltage produced by the stimulator device can be varied based on the desired treatment. For example, the stimulator device can be configured for below sensory stimulation when worn by a patient while sleeping. Below sensory stimulation is when the voltage and/or current is adjusted to be just below where the patient can feel the effects of the electric current. The voltage required for below sensory stimulation varies by the individual depending on skin characteristics. A voltage between about 50 volts and about 100 volts achieves below sensory stimulation for most humans. In example embodiments, a current of between about 20 microamps and about 100 microamps, for example between about 50-80 microamps, can be used. The stimulator device can be configured to run for an interval or intervals of time at one or more levels and/or frequencies of stimulation based on the desired treatment protocol. For example, the stimulator device can be configured for a repeated interval of 20 minutes of electric stimulation and 40 minutes off. The stimulator device can also be configured to vary the level of stimulation over time. For example, the stimulator device can use a logarithmic stimulation protocol. In example embodiments, a treatment protocol delivering pulsed direct current (D.C.) in a twin-peak, mono-phasic wave form is delivered.

The electrotherapeutic stimulator material is formed of an electrically conductive material configured to deliver electrical stimulation from the stimulator device to a body part of the patient. The stimulator material can be formed from panels, strips or other portions of conductive material attached to the inside of the garment. In alternate embodiments, the stimulator material is embedded in the garment material. The remaining garment material is generally not conductive. In example embodiments, the stimulator material is laminated inside the garment material such that the non-conductive garment material provides an insulating layer between the conductive stimulator material and the outside surface of the garment in regions. The insulating garment material layer helps prevent inadvertent contact between the stimulator material and another conductive surface that could cause the stimulator material to ground or short. The stimulator material generally includes a metal or other conductive material including silver, copper, gold or a combination thereof. In example embodiments, the stimulator material is formed from silver conductive panels. The silver conductive panels can be formed in different shapes and configurations based on the desired treatment area. The stimulator material can be formed in compliance with the FDA/GMP as well as the Medical Device Directive of the EU ISO13485/2003. In example embodiments, one or more pairs of stimulator material panels or strips are used with a first strip of stimulator material emitting a positive charge and a second strip of stimulator material emitting a negative charge. Generally the negative electrode is positioned distal to the patient's torso or chest and the positive electrode is positioned proximal to the patient's torso or chest. When utilized on arms, legs, or other extremities, the electrostimulation is preferably delivered with a polarity configured with a polarity configured to position the negative (−) electrode at a distal position on the extremity (i.e., further from the torso) and the positive (+) electrode at a proximal position (closer to the torso). In example embodiments, an electrotherapeutic garment system includes one or more electrotherapeutic garment components (e.g., pants, tights, shirt, stocking, sleeve, mask, patch, band, strip, etc., and/or combinations of any two or more of such components), and each garment component of the electrotherapeutic garment system includes one or more electrodes or sections of electrotherapeutic stimulator material.

FIGS. 1-3 show an example embodiment of a therapeutic tights garment 10 according to an example embodiment of the invention. The tights or pants 10 are configured to cover the legs of the patient. In the example embodiments, each of the tights' legs includes two portions or panels of stimulating material 20. In example embodiments, the panels have a width of about 6 inches. In other embodiments, different size panels can be used. The first stimulating material panel 22 is configured to wrap around the quadriceps of the patient and the second stimulating material panel 24 is configured to wrap around the calf muscle of the patient. The stimulating material panels 20 are generally separated by a portion of non-conductive garment material 12. The circumferential stimulating material panels 20 are configured to go almost all the way around leg. The panels 20 are configured to create an electric field that goes around the leg muscles. In example embodiments, the stimulating material panels 20 extend about 250° to about 270° around the leg muscles. In other embodiments, the stimulating material panels 20 can extend 360° or all the way around the leg muscle. In the example embodiments, the first panel 22 is configured to be the positive pole and the second panel 24 is configured to be the negative pole. In alternate embodiments, the position of the poles can be reversed. In other embodiments, the tights 10 can further include stimulating material configured to be positioned against the gluteal muscles and in particular the gluteus maximus muscles of the patient. The tight garment material 12 is configured to be tight enough to maintain contact between the stimulating material and the legs of the patient while being loose enough to be breathable and comfortable. In other embodiments, for example for delivering electrostimulation therapy to the torso or arms, the stimulating material panels can be configured as a ring electrode encircling 360° entirely around the body portion to be treated or alternatively partially around the treated body part.

In example embodiments, the tights include a gradient compression with the amount of compression being larger at the ankle and lessening as the tights rise further up the leg. In example embodiments, the average compression exerted at the ankle of the tights is about 10 mmHg to about 15 mmHg at the ankle. In other embodiments, the tights can have a compression of between about 10 mmHg and about 40 mmHg at the ankle. In the example embodiment, the pant garment 10 includes two stimulator devices 30, one for each leg portion of the garment. In the depicted embodiment, the stimulator devices 30 are positioned near the waist band of the tights garment. The garment material includes male snaps 32 configured to engage with female receivers on stimulator device 30. The snaps 32 are configured to releasably couple the stimulator device 30 to the garment material 12. In example embodiments, the tights 10 can further include a hook-and-loop material or other fastener in addition to the male snaps 32 to secure the stimulator device 30 to the garment material 12. The snaps 32 are also configured to transfer the electric current produced by the stimulator device to tracers 34 on the inside of the garment material.

The inside of the tights 10 include flat tracers 34 configured to carry the electric current produced by the stimulator devices 30 from the stimulator devices to the stimulator panels 20. The tracers 34 generally include a conductive material 40 covered by a non-conductive, insulating material 42. The conductive material 40 can be formed from a similar material as the stimulator panels 20. The conductive material 40 runs the length of the tracer 34 and is configured to transfer electric current from the male snaps 32 to the stimulator panels 20. The insulating material 42 can be formed of a rubber based silicon or other non-conductive material. The conductive and insulating materials of the tracer 34 can be flexible and washable. The tracers 34 are designed to be substantially flush with the garment material 12. The insulating material of the tracer 34 is positioned between the conductive material of the tracer and the body part of the patient. The tracers 34 are designed to transfer electric current from the stimulator device 30 to the stimulator material 20 without transferring electric current from the tracer to a body part of the patient at intermediate locations where the tracer is covered by the insulating material. In the depicted embodiment, each leg of the tights includes two tracers 34. A first tracer 36 connects the positive pole 22 to the stimulator device 30 and the second tracer 38 connects the negative pole 24 to the stimulator device. In the depicted embodiment, the first and second tracers 36, 38 are separated by non-conductive garment material 12.

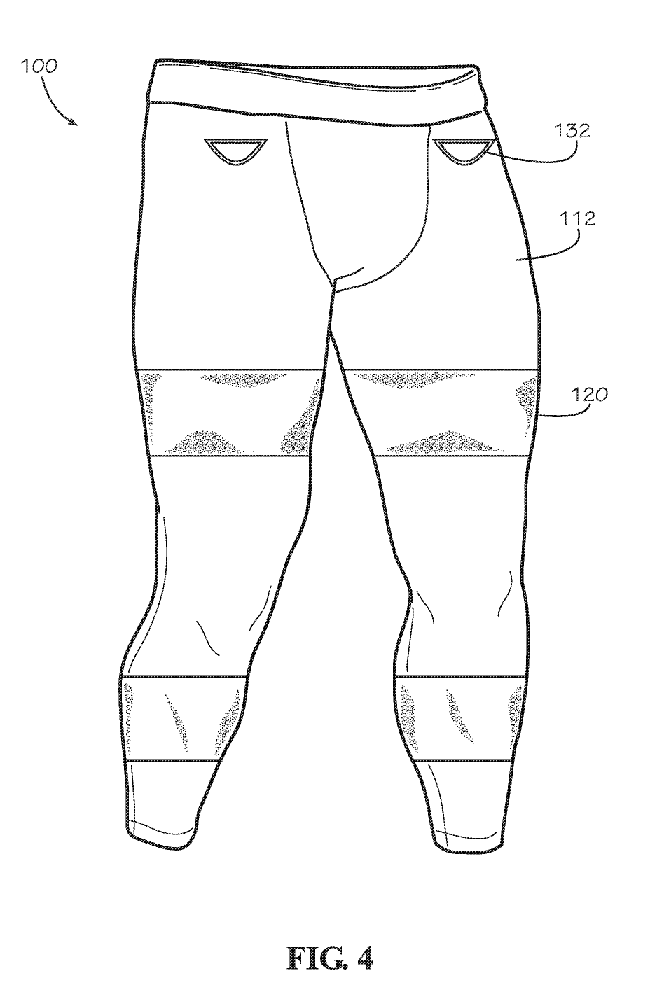

FIG. 4 shows an example embodiment of a therapeutic tights garment 100 according to another example embodiment of the invention. The tights garment 100 includes tights garment material 112 and stimulating material 120. The therapeutic tights 100 can also include docking stations or holsters 132 designed to removably couple the stimulator devices to the tights 100. The holsters or pockets 132 are configured to hold the stimulator devices onto the tights garment such that they engage with stimulating material through leads or tracers. In this example embodiment, the tights material 112 is configured to deliver a higher degree of medical compression than the previous embodiment, for example the tights material 112 can create a compression of about 30 mmHg to about 40 mmHg. The tights 100 include stimulator panels 120 that are about 5 inches in width. In the example embodiments, the tights 100 include lead wires embedded in the garment material 112 to connect the stimulator devices and the stimulating material 120.

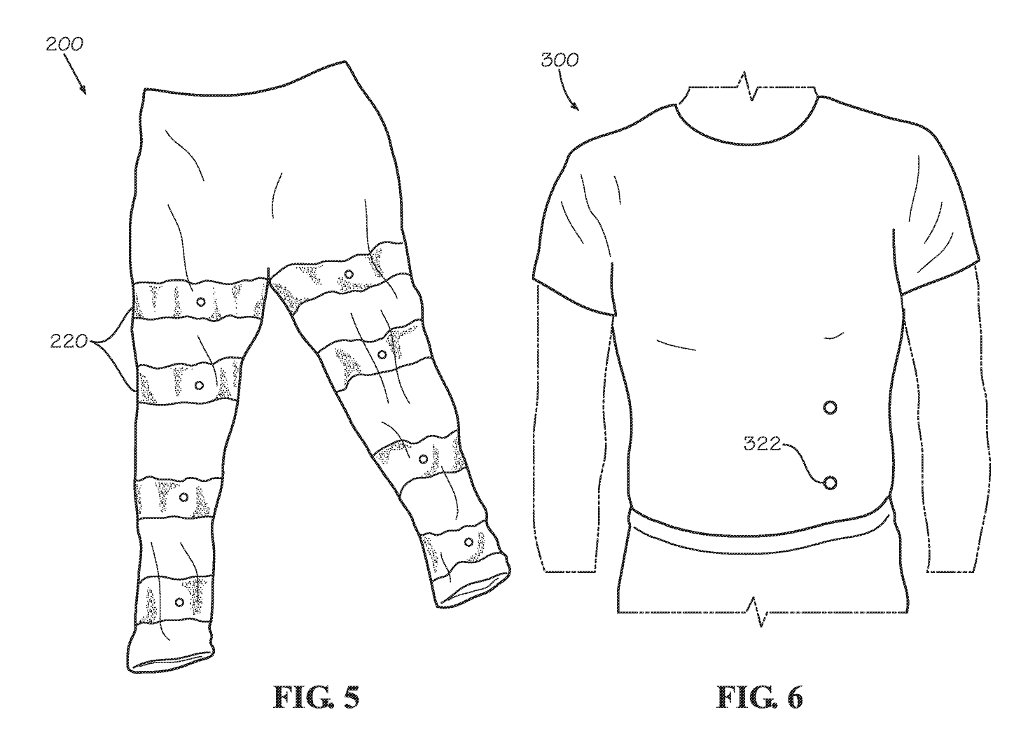

In example embodiments, the therapeutic pant garment 200 includes multiple pairs of stimulating material panels 220, as shown in FIG. 5. In the depicted embodiment, the pant garment 200 includes two stimulator panels 220 configured to wrap around the quadriceps of the patient and two stimulator panels configured to wrap around the calf muscles of the patient.

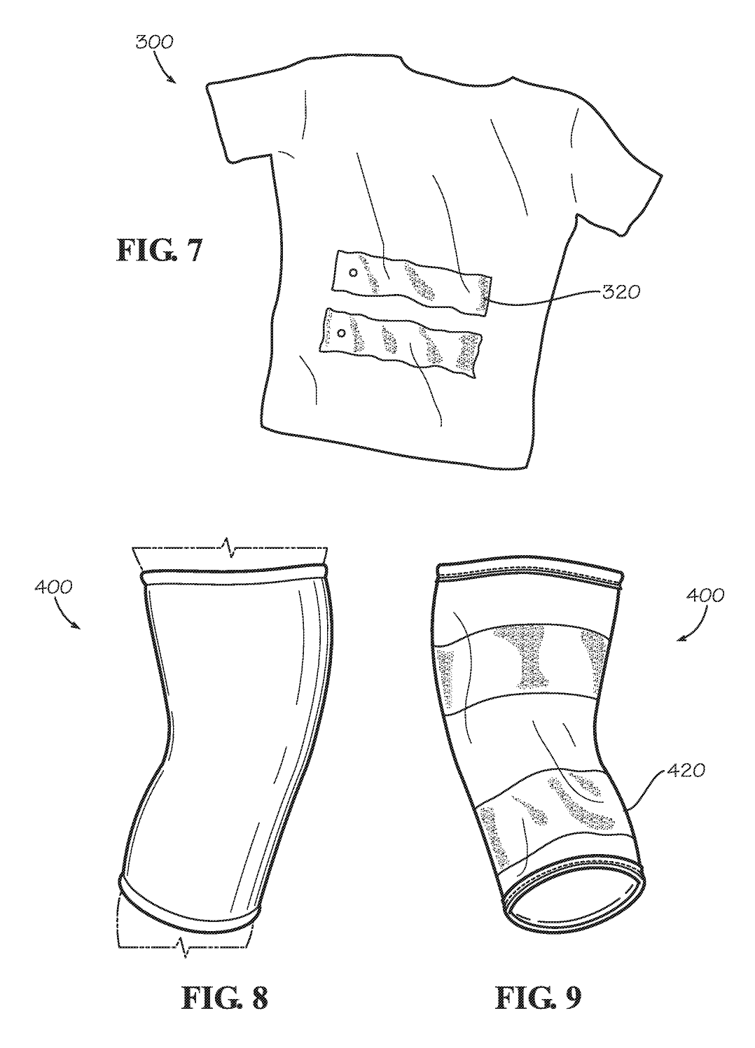

FIGS. 6 and 7 show an example embodiment of a therapeutic shirt garment 300 according to an example embodiment of the invention. The shirt 300 is configured to fit over the chest and torso of a patient. In the example embodiments, the inside of the shirt 300 includes two stimulating material panels 320. The stimulating material panels 320 are configured to be positioned against the abdomen of the patient when the shirt is worn. In other embodiments, the stimulating material panels 320 can be positioned on other portions of the shirt including the back, chest or shoulders. In the example embodiments, the stimulating panels 320 include nodes 322 configured to protrude beyond the outer surface of the shirt. The nodes 322 are configured to engage with a stimulating device, for example through lead wires. In other embodiments, the shirt 300 can include holsters for holding a stimulating device.

FIGS. 8 and 9 show an example of a therapeutic knee sleeve garment 400 according to an example embodiment of the invention. The knee sleeve 400 is configured to fit over the knee of the patient. The knee sleeve 400 can include two or more portions of stimulating material 420. The knee sleeve 400 is designed to hold the stimulating material 420 against the knee area of the patient. The knee sleeve 400 is configured to deliver electrotherapy to the knee of a patient.

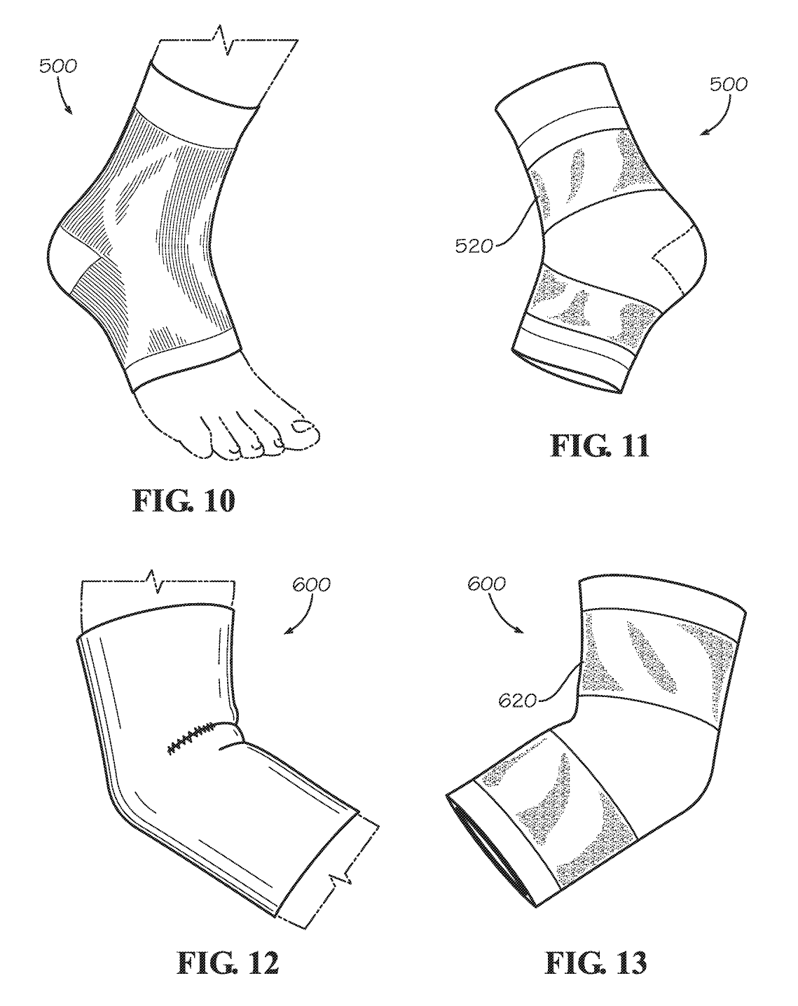

FIGS. 10 and 11 show an example of a therapeutic ankle sleeve garment 500 according to an example embodiment of the invention. The ankle sleeve 500 is configured to fit over the ankle of the patient. The ankle sleeve 500 can include two or more portions of stimulating material 520. The ankle sleeve 600 is designed to hold the stimulating material 520 against the ankle area of the patient. The ankle sleeve is configured to deliver electrotherapy to the ankle of a patient.

FIGS. 12 and 13 show an example of a therapeutic elbow sleeve 600 according to an example embodiment of the invention. The elbow sleeve 600 is configured to fit over the elbow of the patient. The elbow sleeve 600 can include two or more portions of stimulating material 620. The elbow sleeve 600 is designed to hold the stimulating material 620 against the elbow area of the patient. The elbow sleeve 600 is configured to deliver electrotherapy to the elbow of a patient.

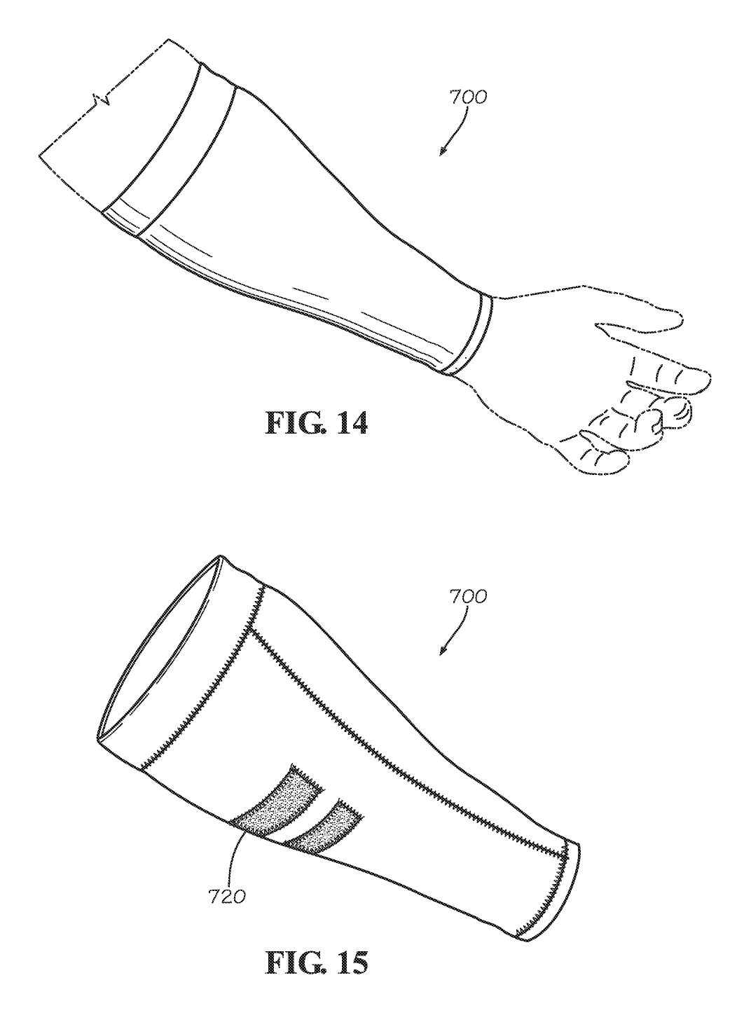

FIGS. 14 and 15 show an example of a therapeutic forearm sleeve 700 according to an example embodiment of the invention. The forearm sleeve 700 is configured to fit over the forearm of the patient. The forearm sleeve 700 can include two or more portions of stimulating material 720. The forearm sleeve 700 is designed to hold the stimulating material 720 against the forearm area of the patient. The forearm sleeve 700 is configured to deliver electrotherapy to the forearm of a patient.

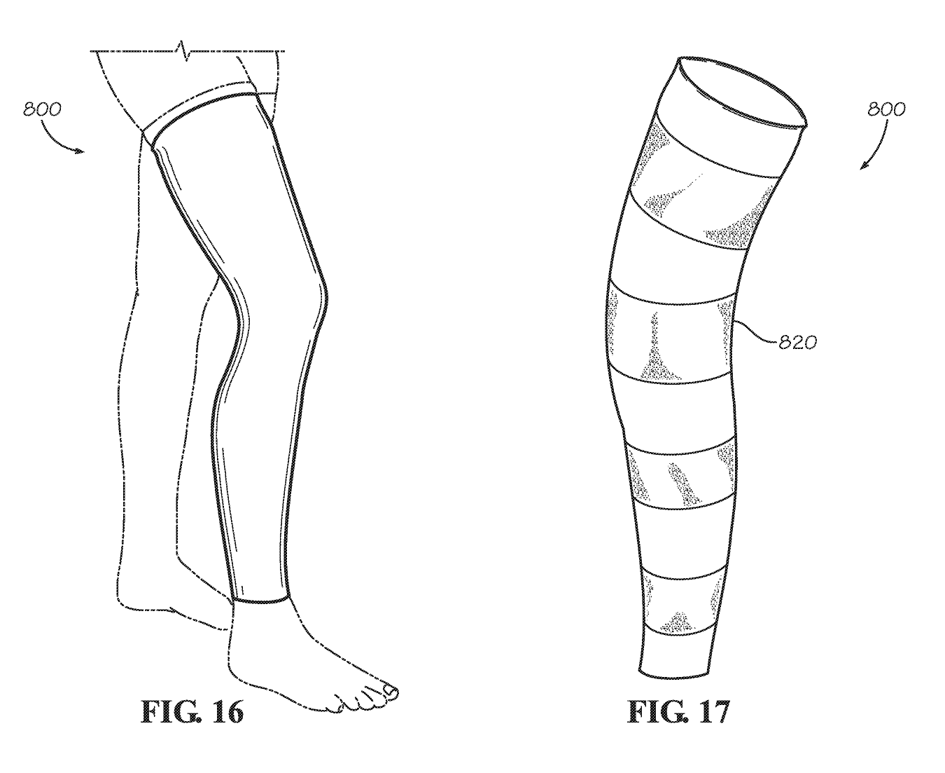

FIGS. 16 and 17 show an example of a therapeutic leg sleeve 800 according to an example embodiment of the invention. The leg sleeve 800 is configured to fit over the leg of the patient. The leg sleeve 800 can include two or more portions of stimulating material 820 positioned similarly to stimulating panels in the therapeutic pant garments. The leg sleeve 800 is designed to hold the stimulating material 820 against the leg of the patient. The leg sleeve 800 is configured to deliver electrotherapy to the leg of a patient.

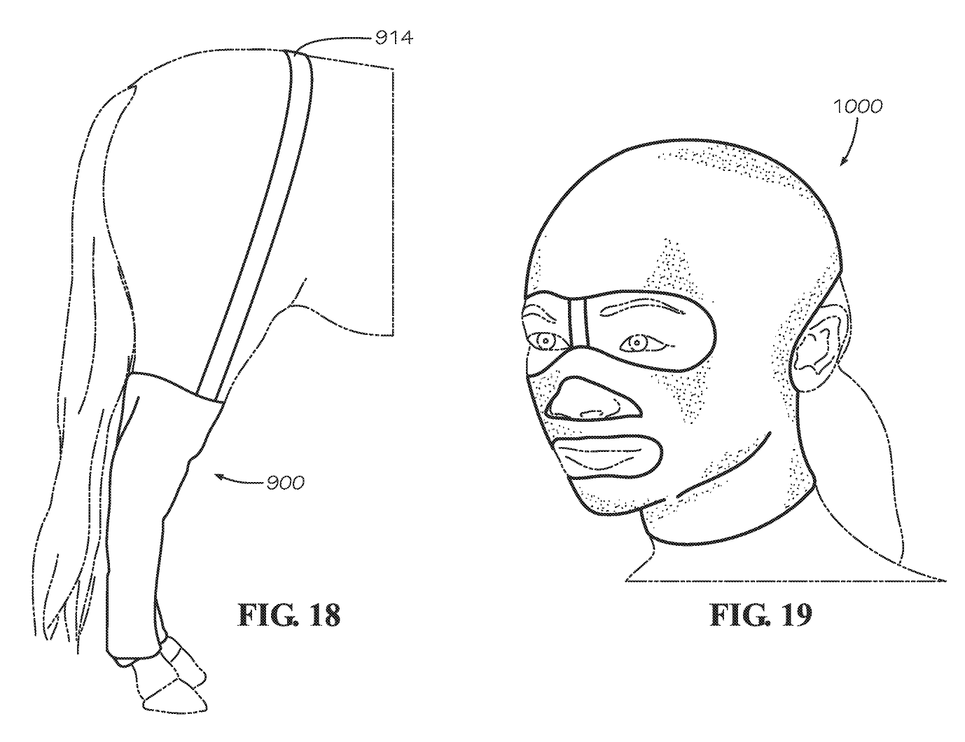

In alternate embodiments, the therapeutic garments can be used to treat animals. In the example embodiment of FIG. 18, a therapeutic leg sleeve 900 is used to deliver electrotherapeutic energy to the legs of a horse. In example embodiments, at least one pair of stimulating material portions is attached to a leg sleeve 900. In alternate embodiments, the garment can be a wrap or stocking. The leg sleeve 900 can also include straps, tape, bands, etc. 914 configured to keep the garment in place on the horse's leg. In example embodiments, the leg sleeve 900 includes two electrodes positioned to engage with the horse's thigh.

FIG. 19 shows a therapeutic face mask 1000 configured for treatment of the face, head and/or neck of a patient. The facial mask 1000 is configured to form a skin tight contact with the wearers face, head and upper neck. The mask includes at least one pair of stimulating material portions configured to engage with a portion of the skin covered by the mask. The stimulating material portions can form an integral part of the mask surface. Alternatively, the stimulating material portions are attached to the interior surface of the mask.

The therapeutic garments can be used in different treatment applications. For example, the therapeutic garment can be worn after exercise or post athletic performance. In example methods of use, the garment is worn for several hours post exercise. In other methods of use, the garment is worn while the patient is sleeping. When used while sleeping, the stimulating device can be configured to emit electric stimulation below sensory levels. To achieve below sensory levels, the patient or wearer can adjust the voltage of the pulsed DC current emitted by the stimulator device until the patient can just feel the effects of the current. Then the patient can lower the voltage in intervals until the patient can no longer feel the effects of the current, but here electrotherapeutic stimulation is still being delivered. In example embodiments, the garment is configured for one or more activities, for example configured as athletic wear for exercise activities, configured as pajamas for rest or sleeping, configured as active casual wear for routine daily activities, and/or configured as business or formal attire for business or social activities. In example embodiments, the cut, size, tightness, elasticity, fabric, colors, and/or other features of the garment are selected depending upon the intended activity or application. For example, placement of the electrodes and/or positioning of the holsters or pockets for retaining a stimulator device and/or other system components may vary depending on the intended use, for example to reduce friction or pressure points when the wearer is standing, seated, laying horizontally, reclined, or otherwise positioned during use of the garment.

While the invention has been described with reference to preferred and example embodiments, it will be understood by those skilled in the art that a variety of modifications, additions and deletions are within the scope of the invention, as defined by the following claims. For example, the stimulator material and device can be used with other garment types including wraps, gloves, and socks.

What is claimed is:

1. An electrotherapeutic garment system for delivering electrotherapy to the body part of a human or animal patient, the electrotherapeutic garment system comprising:
at least one portion of electrotherapeutic stimulating material;
a garment material configured to hold the electrotherapeutic stimulating material in contact with the body part of a patient;
at least one stimulator device configured to produce electrotherapeutic energy; and
at least one conductive tracer configured to transfer the electrotherapeutic energy produced by the at least one stimulating device to the at least one portion of electrotherapeutic material.

2. The electrotherapeutic garment system of claim 1, wherein the garment material is configured as tights.

3. The electrotherapeutic garment system of claim 2, further comprising at least one male snap for detachably coupling with the at least one stimulator device.

4. The electrotherapeutic garment system of claim 2, wherein the garment material further comprises at least one degree of elasticity to produce a compression gradient on at least a portion of the body part to which the garment is applied.

5. The electrotherapeutic garment system of claim 1, wherein the at least one conductive tracer includes an insulating cover.

6. The electrotherapeutic garment system of claim 1, wherein the garment material is configured as a face mask.

7. The electrotherapeutic garment system of claim 1, wherein the garment material is configured to cover an animal appendage.

8. The electrotherapeutic garment system of claim 1, wherein the garment material is configured as a sleeve.

9. The electrotherapeutic garment system of claim 8, wherein the sleeve is configured to cover the leg of a patient.

10. The electrotherapeutic garment system of claim 8, wherein the sleeve is configured to cover the forearm of a patient.

11. The electrotherapeutic garment system of claim 8, wherein the sleeve is configured to cover the elbow of a patient.

12. The electrotherapeutic garment system of claim 8, wherein the sleeve is configured to cover the ankle of a patient.

13. The electrotherapeutic garment system of claim 8, wherein the sleeve is configured to cover the knee of a patient.

14. An electrotherapeutic garment for delivering electrotherapy to a body part of a human or animal patient, the electrotherapeutic garment comprising:
at least one portion of electrotherapeutic stimulating material formed from electro-mesh silver yards;
a garment material;
and at least one stimulating device configured to deliver electrotherapeutic energy to the electrotherapeutic stimulating material.

15. The electrotherapeutic garment of claim 14, wherein the electrotherapeutic stimulating material is attached to the garment material.

16. The electrotherapeutic garment of claim 14, wherein the electrotherapeutic stimulating material is integrally formed with the garment material.

17. The electrotherapeutic garment of claim 14, wherein the garment material is configured to form pants.

18. The electrotherapeutic garment of claim 17, further comprising at least one holster or pocket for receiving the at least one stimulation device

19. The electrotherapeutic garment of claim 14, wherein the garment material is configured to form a sleeve.

20. A method of treating a body portion of a human or animal subject comprising:
applying an electrotherapeutic garment over a body portion of a human or animal subject to be treated; and
delivering therapeutic energy to the body portion through the electrotherapeutic garment.

21. The method of claim 20, wherein the electrotherapeutic garment is worn while the subject is sleeping.
